(19) United States
(12) Patent Application Publication
(10) Pub. No.: US 2025/0128038 A1
(43) Pub. Date: Apr. 24, 2025

(54) MICRONEEDLE STRUCTURE WITH HIGH SEPARATION YIELD, DEVICE CONTAINING THE SAME, AND METHOD FOR MANUFACTURING THE SAME

(71) Applicant: UIF (University Industry Foundation), Yonsei University, Seoul (KR)

(72) Inventors: Won Hyoung RYU, Goyang-si (KR); Ji Yeon YI, Seoul (KR)

(73) Assignee: UIF (University Industry Foundation), Yonsei University, Seoul (KR)

(21) Appl. No.: 18/912,841

(22) Filed: Oct. 11, 2024

(30) Foreign Application Priority Data
Oct. 19, 2023 (KR) 10-2023-0140301

(51) Int. Cl. A61M 37/00

(52) U.S. Cl. CPC A61M 37/0015; A61M 2037/0023; A61M 2037/0053; A61M 2202/0007; A61M 2205/0211; A61M 2205/0216; A61M 2205/0233; A61M 2205/0255; A61M 2207/10

(57) ABSTRACT

A highly-removable microneedle structure, a microneedle device including the same, and a method for manufacturing the structure are disclosed. The highly-removable microneedle structure includes: a tip tapered downwardly; a base extending upwardly from an upper surface of the tip and having an area size in a plan view thereof smaller than an area size in a plan view of an upper surface of the tip; and a tube surrounding the base and extending upwardly from the upper surface of the tip and beyond an upper surface of the base.

### CROSS-REFERENCE TO RELATED APPLICATION

This application claims the benefit of and priority to Korean Patent Application No. 10-2023-0140301 filed on Oct. 19, 2023 in the Korean Intellectual Property Office, and all the benefits accruing therefrom under 35 U.S.C. 119. The contents of the referenced application are incorporated in the present application by reference.

### BACKGROUND

### Field

The present disclosure relates to a highly-removable microneedle structure, a microneedle device including the same, and a method for manufacturing the highly-removable microneedle structure.

In particular, the present disclosure relates to a method for manufacturing a removable microneedle and a microneedle device having the removable microneedle, in which an anchor structure microneedle having a tip and a base is formed in a cylindrical micro tube in a molding manner, and a pushing unit pushes an upper surface of the base of the microneedle such that a combination of the base and the tip is removed from the tube, thereby improving a removal probability of the combination of the microneedle. Further, the removable microneedle may be applied to various living tissues including skin using an individual applicator.

### Description of Related Art

The microneedle has been developed as a device that may be inserted to a position hundreds of micrometers deep from the outermost layer of the living tissue and may deliver a local and highly efficient drug in a minimally invasive manner. Among the microneedles, the removable microneedle is composed of a tip (a drug-loaded portion) having the drug loaded therein and a substrate. The removable microneedle has been used in a manner such that the drug-loaded portion of the microneedle detaches from the substrate after insertion into the skin and releases the drug to a target area quickly or slowly (1020190123642, WO2014150069, WO2014100750, KR101830398B1, Lahiji et al., Scientific reports). In order to apply the microneedle to areas where it is difficult to apply the microneedle system, such as narrow areas, curved areas, or body hairs, it is important to remove the drug-loaded portion from the substrate in an easy and fast manner.

Among studies on removing the drug-loaded portion, studies on removing the same by inserting a sacrificial layer that dissolves into between the drug-loaded portion and the substrate have been conducted (WO2021072313A1). The sacrificial layer that can be dissolved melts the moment the microneedle is inserted into the biological tissue, such that the drug-loaded portion is removed from the substrate. In this way, the drug-loaded portion may be removed reliably. However, this approach has a limitation in that it requires a waiting time until the sacrificial layer has dissolved.

Other methods have been proposed to induce physical removal of the drug-loaded portion from the base by applying an external force to the drug-loaded portion (1020932350000, 1017766590000). Among the methods, a method in which the substrate is deformable such that the drug-loaded portion may be removed from the substrate quickly after insertion into the skin (Jun et al. RSC advances, Chu et al. J control release). In this approach, only 42% of the drug-loaded portion of the microneedle with a substrate shape is inserted into the skin, whereas an inserted portion of the drug-loaded portion of the microneedle with the deformable substrate increases significantly to approximately 80%. In addition, a study is proposed to create a void space between the drug-loaded portion and the substrate and insert the microneedle into the skin, and then apply a shear force so that the drug-loaded portion may be removed from the substrate (Yang et al., Nano Research, Li et al., Nature biomedical engineering, Jung et al., Pharmaceutics). However, this approach has limitations in that the removal occurs probabilistically because the accuracy and repeatability of the void creation are low, and the force application direction change is required when applying an external force to remove the drug-loaded portion, and the user's skillful use is required to apply the external force.

### PRIOR ART LITERATURES

### Patent Literatures

Patent literature 1: 1020932350000_Microneedle and its manufacturing method_Arrowhead.

Patent literature 2: 1020190123642_Multilayer microneedle with excellent skin permeability, patch including same, and method for manufacturing the patch.

Patent literature 3: 1017766590000_Microneedle and its manufacturing method.

Patent literature 4: WO2021072313A1_Silk fibroin-based microneedle and its use.

Patent literature 5: WO2014100750_Microarray for delivery of therapeutic agent and its use method.

Patent literature 6: WO2014150069_Microstructure array for delivering active agent.

Patent literature 7: KR101830398B1_Painless and Patchless Shooting Microstructures.

### Non-Patent Literatures

(Non-patent literature 1) Jun, Hyesun, et al. “Immediate Separation of Microneedle Tips from Base Array during Skin Insertion for Instantaneous Drug Delivery.” RSC Advances, vol. 8, no. 32, 2018, pp. 17786-17796, https://doi.org/10.1039/c8ra02334d.

(Non-patent literature 2) Yang, Yuan, et al. “Rapidly Separable Bubble Microneedle Patch for Effective Local Anesthesia.” Nano Research, vol. 15, no. 9, 2022, pp. 8336-8344, https://doi.org/10.1007/s12274-022-4508-y.

(Non-patent literature 3) Lee, KangJu, et al. “Intracorneal Injection of a Detachable Hybrid Microneedle for Sustained Drug Delivery.” Acta Biomaterialia, vol. 80, 2018, pp. 48-57, https://doi.org/10.1016/j.actbio.2018.09.039.

(Non-patent literature 4) Li, Wei, et al. “Rapidly Separable Microneedle Patch for the Sustained Release of a Contraceptive.” Nature Biomedical Engineering, vol. 3, no. 3, 2019, pp. 220-229, https://doi.org/10.1038/s41551-018-0337-4.

(Non-patent literature 5) Jung, Chung-ryong, et al. “Rapidly Separable Micropillar Integrated Dissolving Microneedles.” Pharmaceutics, vol. 12, no. 6, 2020, p. 581, https://doi.org/10.3390/pharmaceutics12060581.

(Non-patent literature 6) Chu, Leonard Y., and Mark R. Prausnitz. “Separable Arrowhead Microneedles.” Journal of Controlled Release, vol. 149, no. 3, 2011, pp. 242-249, https://doi.org/10.1016/j.jconrel.2010.10.033.

(Non-patent literature 7) Lahiji, Shayan F., et al. “A patchless dissolving microneedle delivery system enabling rapid and efficient transdermal drug delivery.” Scientific Reports, vol. 5, no. 1, 2015, https://doi.org/10.1038/srep07914.

### SUMMARY

One purpose of the present disclosure is to provide a highly-removable microneedle structure including an anchor structure in which a drug-loaded portion is removed at a high probability when an applicator is removed after penetration into the skin.

Another purpose of the present disclosure is to provide a microneedle device including the highly-removable microneedle structure, in which when the device is applied to the skin, the drug-loaded portion is removed at a high probability.

Still another purpose of the present disclosure is to provide a method for manufacturing the highly-removable microneedle structure.

In particular, a purpose of the present disclosure is to increase the removal probability of the drug-loaded portion from the substrate in a conventional removable microneedle and to provide a removal system for the removable microneedle structure.

Purposes according to the present disclosure are not limited to the above-mentioned purpose. Other purposes and advantages according to the present disclosure that are not mentioned may be understood based on following descriptions, and may be more clearly understood based on embodiments according to the present disclosure. Further, it will be easily understood that the purposes and advantages according to the present disclosure may be realized using means shown in the claims or combinations thereof.

A first aspect of the present disclosure provides a highly-removable microneedle structure comprising: a tip tapered downwardly; a base extending upwardly from an upper surface of the tip and having an area size in a plan view thereof smaller than an area size in a plan view of an upper surface of the tip; and a tube surrounding the base and extending upwardly from the upper surface of the tip and beyond an upper surface of the base.

In accordance with some embodiments of the highly-removable microneedle structure, the base is fitted into the tube.

In accordance with some embodiments of the highly-removable microneedle structure, the tip or the base includes a delivery target drug.

In accordance with some embodiments of the highly-removable microneedle structure, the tip or the base includes at least one selected from a group including a thermoplastic polymer; a photocurable polymer; a biocompatible polysaccharide polymer including hyaluronic acid, chitosan, and alginic acid; and a natural biomolecule including a protein or DNA.

In accordance with some embodiments of the highly-removable microneedle structure, the tube includes at least one selected from a group including a polymer including PTFE, a metal, and a ceramic.

A second aspect of the present disclosure provides a microneedle device having a highly-removable microneedle structure, the microneedle device comprising: a microneedle structure including: a tip tapered downwardly; a base extending upwardly from an upper surface of the tip and having an area size in a plan view thereof smaller than an area size in a plan view of an upper surface of the tip; and a tube surrounding the base and extending upwardly from the upper surface of the tip and beyond an upper surface of the base; and a microneedle pushing unit including: a push pin extending upwardly from the upper surface of the base and beyond an upper surface of the tube; and a pushing means capable of applying a pushing force to an upper end of the push pin, wherein the microneedle device is configured such that when the pushing means applies the force to the upper end of the push pin, a lower end of the push pin applies the force to the base, so that a combination of the tip and the base is removed from the tube, the lower end of the push pin fixes the combination of the tip and the base into a skin, so that the tube is easily removed from the combination.

In accordance with some embodiments of the microneedle device having the highly-removable microneedle structure, wherein the base is fitted into the tube.

In accordance with some embodiments of the microneedle device having the highly-removable microneedle structure, wherein the tip or the base includes a delivery target drug.

In accordance with some embodiments of the microneedle device having the highly-removable microneedle structure, wherein the tip or the base includes at least one selected from a group including a thermoplastic polymer; a photocurable polymer; a biocompatible polysaccharide polymer including hyaluronic acid, chitosan, and alginic acid; and a natural biomolecule including a protein or DNA.

In accordance with some embodiments of the microneedle device having the highly-removable microneedle structure, wherein the tube includes at least one selected from a group including a polymer including PTFE, a metal, and a ceramic.

In accordance with some embodiments of the microneedle device having the highly-removable microneedle structure, wherein the pushing means applies the force to the push pin under at least one of a force from a human hand, an electromagnetic force, a spring force, a pneumatic pressure, and a hydraulic pressure.

A third aspect of the present disclosure provides a method for manufacturing a highly-removable microneedle structure, the method comprising: providing a mold a recess defined therein, the recess having a tip shape tapered downwardly; filling a curable delivery target substance solution into the recess; providing a tube having an inner diameter smaller than an outer diameter of an upper surface of the tip shape; filling the delivery target substance solution into one end of the tube; and contacting the one end of the tube with an upper surface of the delivery target substance solution received in the recess while closing the other end of the tube or applying a negative pressure to the other end of the tube.

In accordance with some embodiments of the method for manufacturing the highly-removable microneedle structure, the delivery target substance solution includes a delivery target drug.

In accordance with some embodiments of the method for manufacturing the highly-removable microneedle structure, the delivery target substance solution includes at least one selected from a group including a thermoplastic polymer; a photocurable polymer; a biocompatible polysaccharide polymer including hyaluronic acid, chitosan, and alginic acid; and a natural biomolecule including a protein or DNA, wherein the delivery target substance solution is subjected to heat and thus is provided in a fluid state in which the delivery target substance solution can fill the recess.

In accordance with some embodiments of the method for manufacturing the highly-removable microneedle structure, the tube includes at least one selected from a group including a polymer including PTFE, a metal, and a ceramic.

According to the present disclosure, the highly-removable microneedle structure may maximize the removal probability of the drug-loaded portion using the anchor structure after having penetrated the skin.

The microneedle device having the highly-removable microneedle structure according to the present disclosure may apply the microneedle to the skin at high removal probability of the drug-loaded portion.

The method for manufacturing the highly-removable microneedle structure according to the present disclosure may simply and quickly manufacture the highly-removable microneedle structure.

In particular, the present disclosure provides the method for manufacturing the microneedle with an anchor structure in which the drug-loaded portion may be inserted into the tissue and may be removed from the substrate at the high probability, and a removal system using a power source. Accordingly, unlike the conventional approaches, the drug-loaded portion having the anchor shape is manufactured by molding the drug-loaded portion into the end of the microtube and may be physically pushed from a rear surface of the microtube using one or more of a human hand force, electromagnetic force, spring force, pneumatic pressure, and hydraulic pressure to insert the drug-loaded portion into the tissue and then remove the same from the applicator. Therefore, the insertion and removal of the microneedle drug-loaded portion may be achieved immediately without decomposition using water, chemical action, or additional physical force after the insertion, and without a waiting time, and the removal probability of the microneedle drug-loaded portion from the applicator is increased, so that the improvement of drug delivery may be achieved. In particular, the application of the single microneedle drug-loaded portion to a living tissue allows for both ultra-precision application to a local area or application to a wider area in a form of an array.

Effects of the present disclosure are not limited to the effects mentioned above, and other effects not mentioned will be clearly understood by those skilled in the art from the description below.

In addition to the above effects, specific effects of the present disclosure are described together while describing specific details for carrying out the present disclosure.

### BRIEF DESCRIPTION OF DRAWINGS

FIG. 1 is a schematic diagram of a microneedle structure according to an embodiment of the present disclosure.

FIG. 2 is a schematic diagram of a microneedle device according to an embodiment of the present disclosure.

FIG. 3 is a conceptual diagram of a removal system of a removable anchor structure microneedle according to one embodiment of the present disclosure.

FIG. 4 is a conceptual diagram of a manufacturing process of a removable anchor structure microneedle according to one embodiment of the present disclosure.

FIG. 5A shows a process of manufacturing a drug-loaded portion of a removable anchor structure microneedle using poly (lactic-co-glycolic acid) (PLGA) as a material of the drug-loaded portion in accordance with one embodiment.

FIG. 5B shows a drug-loaded portion manufactured using hyaluronic acid (HA) as the material of the drug-loaded portion in accordance with another embodiment.

FIGS. 6A to 6D shows that a removable microneedle used in one embodiment of the present disclosure is manufactured using a pressure-assisted transfer molding technique using a microtube.

FIGS. 7A to 7D shows a photograph of manufacturing the microneedle structure according to an embodiment of the present disclosure.

FIGS. 8A to 8B shows a photograph of the anchor structure of the microneedle structure according to an embodiment of the present disclosure.

FIG. 9 shows a diagram showing a removal applicator and a process in which a removable anchor structure microneedle of the present disclosure is pushed away from the applicator.

FIGS. 10A to 10B shows a diagram showing the removal of the removable anchor structure microneedle according to one embodiment of the present disclosure using the removal applicator.

### DETAILED DESCRIPTIONS

Advantages and features of the present disclosure, and a method of achieving the advantages and features will become apparent with reference to embodiments described later in detail together with the accompanying drawings. However, the present disclosure is not limited to the embodiments as disclosed under, but may be implemented in various different forms. Thus, these embodiments are set forth only to make the present disclosure complete, and to completely inform the scope of the present disclosure to those of ordinary skill in the technical field to which the present disclosure belongs, and the present disclosure is only defined by the scope of the claims.

For simplicity and clarity of illustration, elements in the drawings are not necessarily drawn to scale. The same reference numbers in different drawings represent the same or similar elements, and as such perform similar functionality. Further, descriptions and details of well-known steps and elements are omitted for simplicity of the description. Furthermore, in the following detailed description of the present disclosure, numerous specific details are set forth in order to provide a thorough understanding of the present disclosure. However, it will be understood that the present disclosure may be practiced without these specific details. In other instances, well-known methods, procedures, components, and circuits have not been described in detail so as not to unnecessarily obscure aspects of the present disclosure. Examples of various embodiments are illustrated and described further below. It will be understood that the description herein is not intended to limit the claims to the specific embodiments described. On the contrary, it is intended to cover alternatives, modifications, and equivalents as may be included within the spirit and scope of the present disclosure as defined by the appended claims.

A shape, a size, a ratio, an angle, a number, etc. disclosed in the drawings for illustrating embodiments of the present disclosure are illustrative, and the present disclosure is not limited thereto.

The terminology used herein is directed to the purpose of describing particular embodiments only and is not intended to be limiting of the present disclosure. As used herein, the singular constitutes “a” and “an” are intended to include the plural constitutes as well, unless the context clearly indicates otherwise. It will be further understood that the terms “comprise”, “comprising”, “include”, and “including” when used in this specification, specify the presence of the stated features, integers, operations, elements, and/or components, but do not preclude the presence or addition of one or more other features, integers, operations, elements, components, and/or portions thereof. As used herein, the term “and/or” includes any and all combinations of one or more of associated listed items. Expression such as “at least one of” when preceding a list of elements may modify the entire list of elements and may not modify the individual elements of the list. In interpretation of numerical values, an error or tolerance therein may occur even when there is no explicit description thereof.

In addition, it will also be understood that when a first element or layer is referred to as being present “on” a second element or layer, the first element may be disposed directly on the second element or may be disposed indirectly on the second element with a third element or layer being disposed between the first and second elements or layers. It will be understood that when an element or layer is referred to as being “connected to”, or “connected to” another element or layer, it may be directly on, connected to, or connected to the other element or layer, or one or more intervening elements or layers may be present. In addition, it will also be understood that when an element or layer is referred to as being “between” two elements or layers, it may be the only element or layer between the two elements or layers, or one or more intervening elements or layers may also be present.

Further, as used herein, when a layer, film, region, plate, or the like is disposed “on” or “on a top” of another layer, film, region, plate, or the like, the former may directly contact the latter or still another layer, film, region, plate, or the like may be disposed between the former and the latter. As used herein, when a layer, film, region, plate, or the like is directly disposed “on” or “on a top” of another layer, film, region, plate, or the like, the former directly contacts the latter and still another layer, film, region, plate, or the like is not disposed between the former and the latter. Further, as used herein, when a layer, film, region, plate, or the like is disposed “below” or “under” another layer, film, region, plate, or the like, the former may directly contact the latter or still another layer, film, region, plate, or the like may be disposed between the former and the latter. As used herein, when a layer, film, region, plate, or the like is directly disposed “below” or “under” another layer, film, region, plate, or the like, the former directly contacts the latter and still another layer, film, region, plate, or the like is not disposed between the former and the latter.

In descriptions of temporal relationships, for example, temporal precedent relationships between two events such as “after”, “subsequent to”, “before”, etc., another event may occur therebetween unless “directly after”, “directly subsequent” or “directly before” is not indicated.

When a certain embodiment may be implemented differently, a function or an operation specified in a specific block may occur in a different order from an order specified in a flowchart. For example, two blocks in succession may be actually performed substantially concurrently, or the two blocks may be performed in a reverse order depending on a function or operation involved.

It will be understood that, although the terms “first”, “second”, “third”, and so on may be used herein to describe various elements, components, regions, layers and/or periods, these elements, components, regions, layers and/or periods should not be limited by these terms. These terms are used to distinguish one element, component, region, layer or section from another element, component, region, layer or period. Thus, a first element, component, region, layer or section as described under could be termed a second element, component, region, layer or period, without departing from the spirit and scope of the present disclosure.

When an embodiment may be implemented differently, functions or operations specified within a specific block may be performed in a different order from an order specified in a flowchart. For example, two consecutive blocks may actually be performed substantially simultaneously, or the blocks may be performed in a reverse order depending on related functions or operations.

The features of the various embodiments of the present disclosure may be partially or entirely combined with each other, and may be technically associated with each other or operate with each other. The embodiments may be implemented independently of each other and may be implemented together in an association relationship.

It will be understood that when an element or layer is referred to as being “connected to”, or “connected to” another element or layer, it may be directly on, connected to, or connected to the other element or layer, or one or more intervening elements or layers may be present. In addition, it will also be understood that when an element or layer is referred to as being “between” two elements or layers, it may be the only element or layer between the two elements or layers, or one or more intervening elements or layers may also be present.

Unless otherwise defined, all terms including technical and scientific terms used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

As used herein, “embodiments,” “examples,” “aspects, and the like should not be construed such that any aspect or design as described is superior to or advantageous over other aspects or designs.

Further, the term ‘or’ means ‘inclusive or’ rather than ‘exclusive or’. That is, unless otherwise stated or clear from the context, the expression that ‘x uses a or b’ means any one of natural inclusive permutations.

The terms used in the description below have been selected as being general and universal in the related technical field. However, there may be other terms than the terms depending on the development and/or change of technology, convention, preference of technicians, etc. Therefore, the terms used in the description below should not be understood as limiting technical ideas, but should be understood as examples of the terms for illustrating embodiments.

Further, in a specific case, a term may be arbitrarily selected by the applicant, and in this case, the detailed meaning thereof will be described in a corresponding description period. Therefore, the terms used in the description below should be understood based on not simply the name of the terms, but the meaning of the terms and the contents throughout the Detailed Descriptions.

Throughout the present disclosure, “A and/or B” means A, B, or A and B, unless otherwise specified, and “C to D” means C inclusive to D inclusive unless otherwise specified.

“At least one” should be understood to include any combination of one or more of listed components. For example, at least one of first, second, and third components means not only a first, second, or third component, but also all combinations of two or more of the first, second, and third components.

Hereinafter, embodiments of the present disclosure will be described using the attached drawings. A scale of each of components as shown in the drawings is different from an actual scale thereof for convenience of illustration, and therefore, the present disclosure not limited to the scale as shown in the drawings.

In interpreting a numerical value, the value is interpreted as including an error range unless there is no separate explicit description thereof. In the context of the present disclosure, the term “about” may mean about ±1%, about ±2%, about ±3%, about ±4%, about ±5%, about ±6%, about ±7%, about ±8%, about ±9%, or about ±10% of the numerical value as recited herein.

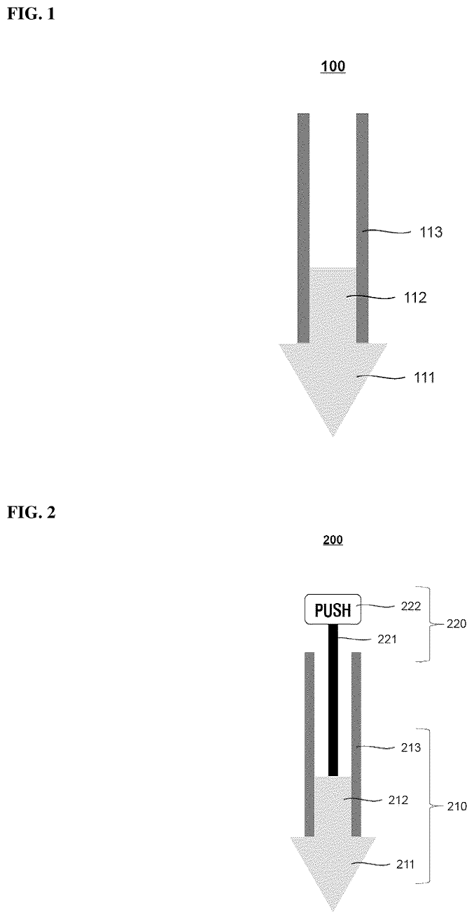

FIG. 1 is a schematic diagram of a microneedle structure according to an embodiment of the present disclosure

Referring to FIG. 1, a microneedle structure 100 according to an embodiment of the present disclosure may have a tip 111 tapered downwardly; a base 112 extending upwardly from an upper surface of the tip 111 and having a smaller area in a plan view than that of the upper surface of the tip 111; and a tube 113 that surrounds the base 112 and extends upwardly from the upper surface of the tip 111 and beyond an upper surface of the base 112.

The tip 111 is a member that provides a pointed end such that the microneedle of the microneedle structure 100 can penetrate the skin. In the context of the present disclosure, the “tip” refers to a member having a shape having a pointed end.

The base 112 is a portion extending upwardly from the tip 111 and has a smaller area in a plan view than that of the upper surface of the tip 111.

The base 112 extends upwardly from the tip 111 and has a smaller area in a plan view than that of the upper surface of the tip 111, such that a combination of the tip and the base constitutes an anchor-like structure. Thus, a force required for the combination of the tip 111 and the base 112 to move in a direction so as to be removed from the skin (i.e., upwardly in this example) after having penetrated the skin may be significantly greater than a force required for the combination of the tip 111 and the base 112 to move in a direction so as to penetrate the skin (i.e., downwardly in this example). This prevents the combination of the base 112 and the tip 111 from being easily removed from the skin after the combination has penetrated the skin. Under the same principle, when another member such as a member such as the tube 113 as described below is coupled to the combination of the tip 111 and the base 112, a force required for the combination of the tip 111 and the base 112 to move in a direction so as to be removed from the skin (i.e., upwardly in this example) after having penetrated the skin may be significantly greater than a force required for the tube 113 to be removed from the combination. Thus, the microneedle with improved removal probability, that is, the highly-removable microneedle may be realized.

The tube 113 is a member that surrounds at least the base 112 and extends upwardly. The tube 113 extends upwardly from the upper surface of the tip 111 and beyond the upper surface of the base 112. That is, a vertical length of the base 112 is smaller than that of the tube 113. Thus, when the base 112 is pushed downwardly using pushing means received in a hollow of the tube 113, the base 112 may be removed from the tube 113. In the context of the present disclosure, “pushing” refers to an act of applying a force to cause an object to move in one-way direction, or a function of a member of applying a force to cause an object to move in one-way direction.

That is, the base 112 and the tip 111 may constitute the anchor structure. Further, the tube 113 surrounding the base 112 and extending upwardly from the upper surface of the tip 111 and beyond the upper surface of the base 112 may be used together with the pushing means to push the combination of the tip 111 and the base 112 downwardly. The base 112 should be stably connected to the tube 113 such that the base can penetrate the skin before the base is removed from the tube 113 after the base has successfully invaded the skin. To this end, a coupling force or a frictional force of a significant level or greater should act between the base 112 and the tube 113.

In one embodiment, the base 112 may be fitted into the tube 113. In the context of the present disclosure, “being fitted into” is not necessarily limited to “being forcibly fitted into”. However, “being fitted into” may mean that a state in which a member has been inserted into another member is maintained substantially under a frictional force. For example, a solution flows into the tube 113, a mold, or a container so as to at least partially fill an inner space defined therein, and so as to come into contact with an inner wall thereof. In this state, the solution may be cured such that the cured product and the inner wall are in close contact with each other. This may not be interpreted not as “being forcibly fitted into”, but as “being fitted into”.

Since the tip 111 and/or the base 112 are maintained in the penetrating state into the skin as described above, the tip 111 and/or the base 112 may include a drug, a biocompatible polymer, a biodegradable polymer, etc. In one embodiment, the tip 111 or the base 112 may include a delivery target drug. In one embodiment, the tip 111 or the base 112 may include a thermoplastic polymer. It will be apparent from the description as set forth below that when the tip 111 or the base 112 is made of the thermoplastic polymer, it is easy to manufacture the anchor structure composed of the tip 111 and the base 112 and achieve the state in which the base 112 has been fitted into the tube 113.

In one embodiment, the tube 113 may include a polymer tube such as a PTFE tube. The material of the tube 113 is not necessarily limited thereto. As described above, the tip 111 or the base 112 has thermoplasticity. Thus, when the tip 111 or the base 112 is heated into a fluid state, the material of the tube may be at least capable of withstanding the heat.

FIG. 2 schematically illustrates a microneedle device according to an embodiment of the present disclosure.

Referring to FIG. 2, a microneedle device 200 according to an embodiment of the present disclosure may include a microneedle structure 210; and a microneedle pushing unit 220 including a push pin 221 extending upwardly from an upper surface of a base 212 and beyond an upper surface of a tube 213; and a pushing means 222 capable of applying a pushing force to an upper end of the push pin 221.

The description of the microneedle structure according to one aspect of the present disclosure may be applied identically or similarly to the same or similar configuration or term in the description of the microneedle device according to another aspect thereof. Thus, redundant descriptions will be omitted.

The microneedle structure 210 may be identical with the microneedle structure 100 according to an embodiment of the present disclosure. That is, the microneedle structure 210 may have a tip 211 tapered downwardly; the base 212 extending upwardly from an upper surface of the tip 211 and having a smaller area in a plan view than that of the upper surface of the tip 211; and the tube 213 that surrounds the base 212 and extends upwardly from the upper surface of the tip 211 and beyond an upper surface of the base 212.

In one embodiment, the microneedle device 200 having the highly-removable microneedle structure 210 may be provided, wherein when the pushing means 222 applies the force to one end of the push pin 221, the other end of the push pin 221 applies the force to the base 212, thereby causing the combination of the tip 211 and the base 212 to be removed from the tube 213.

The base 212 extends upwardly from the tip 211 and has a smaller area in a plan view than that of the upper surface of the tip 211, such that a combination of the tip and the base constitutes an anchor-like structure. Thus, a force required for the combination of the tip 211 and the base 212 to move in a direction so as to be removed from the skin (i.e., upwardly in this example) after having penetrated the skin may be significantly greater than a force required for the combination of the tip 211 and the base 212 to move in a direction so as to penetrate the skin (i.e., downwardly in this example). This prevents the combination of the base 212 and the tip 211 from being easily removed from the skin after the combination has penetrated the skin. Under the same principle, when another member such as a member such as the tube 213 as described below is coupled to the combination of the tip 211 and the base 212, a force required for the combination of the tip 211 and the base 212 to move in a direction so as to be removed from the skin (i.e., upwardly in this example) after having penetrated the skin may be significantly greater than a force required for the tube 213 to be removed from the combination. Thus, the microneedle with improved removal probability, that is, the highly-removable microneedle may be realized.

The tube 213 is a member that surrounds at least the base 212 and extends upwardly. The tube 213 extends upwardly from the upper surface of the tip 211 and beyond the upper surface of the base 212. That is, a vertical length of the base 212 is smaller than that of the tube 213. Thus, when the base 212 is pushed downwardly using pushing means received in a hollow of the tube 213, the base 212 may be removed from the tube 213.

That is, the base 212 and the tip 211 may constitute the anchor structure. Further, the tube 213 surrounding the base 212 and extending upwardly from the upper surface of the tip 211 and beyond the upper surface of the base 212 may be used together with the pushing means to push the combination of the tip 211 and the base 212 downwardly. The base 212 should be stably connected to the tube 213 such that the base can penetrate the skin before the base is removed from the tube 213 after the base has successfully invaded the skin. To this end, a coupling force or a frictional force of a significant level or greater should act between the base 212 and the tube 213.

Since the tip 211 and/or the base 212 are maintained in the penetrating state into the skin as described above, the tip 211 and/or the base 212 may include a drug, a biocompatible polymer, a biodegradable polymer, etc. In one embodiment, the tip 211 or the base 212 may include a delivery target drug. In one embodiment, the tip 211 or the base 212 may include a thermoplastic polymer. It will be apparent from the description as set forth below that when the tip 211 or the base 212 is made of the thermoplastic polymer, it is easy to manufacture the anchor structure composed of the tip 211 and the base 212 and achieve the state in which the base 212 has been fitted into the tube 213.

In one embodiment, the tube 213 may include a polymer tube such as a PTFE tube. The material of the tube 213 is not necessarily limited thereto. As described above, the tip 211 or the base 212 has thermoplasticity. Thus, when the tip 211 or the base 212 is heated into a fluid state, the material of the tube may be at least capable of withstanding the heat.

The pushing means 222 is not particularly limited in terms of a structure and a shape, or a pushing manner thereof as long as the pushing means 222 may apply the force to the push pin 221. In one embodiment, the pushing means 222 may apply, to the push pin 221, at least one of a force of human hand, an electromagnetic force, a spring force, a pneumatic force, and a hydraulic pressure.

Further, a method for manufacturing a microneedle structure according to the present disclosure may include a first step of providing a mold having a recess having a tip shape tapered downwardly defined therein, and filling a curable delivery target substance solution into the recess; and a second step of providing a tube having an inner diameter smaller than a width of an upper surface of the tip shape, and filling the delivery target substance solution into one end of the tube, and contacting one end of the tube with an upper surface of the delivery target substance solution received in the mold while closing the other end of the tube or applying a negative pressure to the other end of the tube.

The first step is a step of forming the tip, and the second step is a step of forming the base, wherein the tube is formed to extend from the upper surface of the tip while the tube surrounds the base. When the first and second steps as described above are performed, the method for manufacturing the microneedle structure according to an embodiment of the present disclosure may manufacture the highly-removable microneedle structure at a high reliability.

The description of the microneedle structure according to one aspect of the present disclosure may be applied identically or similarly to the same or similar configuration or term in the description of the method for manufacturing the microneedle structure according to still another aspect thereof. Thus, redundant descriptions will be omitted.

Therefore, in one embodiment, the delivery target substance solution may include the delivery target drug. In one embodiment, the delivery target substance solution may include a thermoplastic polymer. In one embodiment, the delivery target substance solution may be subjected to the heat so as to be brought into a fluid state in which the solution may fill the mold or the tube. In one embodiment, the tube may include a polymer tube such as a PTFE tube.

The following describes examples of the present disclosure. However, the examples as described below are only some embodiments of the present disclosure, and the scope of the present disclosure is not limited to the examples as set forth below.

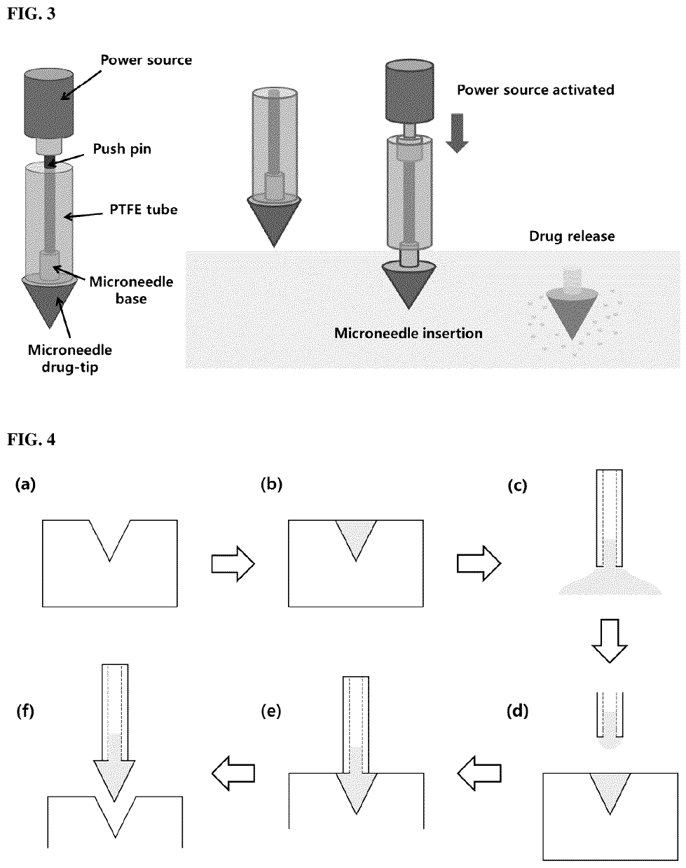

FIG. 3 is a conceptual diagram of a removal system of a removable anchor structure microneedle according to one embodiment of the present disclosure.

FIG. 4 is a conceptual diagram of a fabrication process of the removable anchor structure microneedle according to one embodiment of the present disclosure. As shown in (a) in FIG. 4 (top left) and (b) in FIG. 4 (top center,) a drug-loaded portion is first fabricated. Then, as shown in (c) in FIG. 4 (top right), (d) in FIG. 4 (bottom right), and (e) in FIG. 4 (bottom center), the drug-loaded portion and the base are in contact with each other in the cured manner using the pressure-assisted transfer molding method as mentioned above to fabricate the anchor structure microneedle in the microtube. The fabricated removable anchor structure microneedle has a shape shown in (f) in FIG. 4 (bottom left).

As mentioned in FIG. 4, the drug-loaded portion of the microneedle is first fabricated in fabricating the removable anchor structure microneedle of the present disclosure. To fabricate the drug-loaded portion, PDMS is poured into a relief three-dimensional structure master mold and was cured to fabricate a PDMS microneedle mold having the engraving or the recess as shown in (a) in FIG. 4.

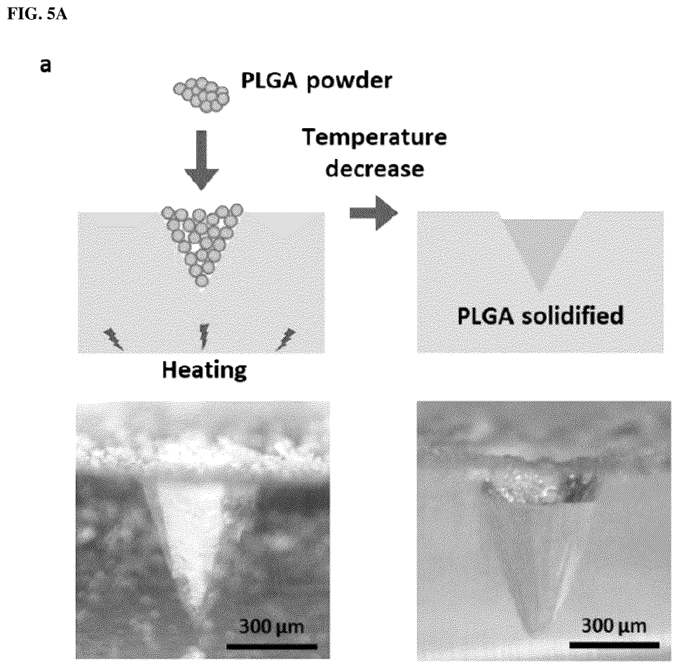

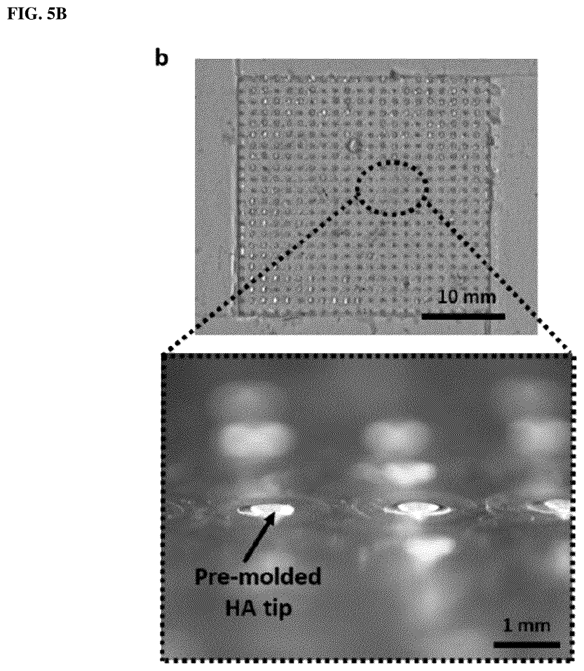

FIG. 5A shows the process of fabricating the drug-loaded portion using poly (lactic-co-glycolic acid) (PLGA) as a material of the drug-loaded portion of the removable anchor structure microneedle in one embodiment. The PLGA is a polymer with thermoplastic properties. When the PLGA is heated to a level above a predefined temperature, it changes into a shapable phase. When the temperature is lowered to a level below a predefined temperature, it returns to a solid state in the shaped state. Using this property of the PLGA, the PLGA powders are filled into the recess or the engraving defined in the PDMS mold, melted under application of heat, and then solidified by lowering the temperature to fill approximately 80% of the mold recess or engraving to fabricate the drug-loaded portion. In addition to PLGA used in one embodiment of the present disclosure, various thermoplastic polymers may be employed.

Examples of various thermoplastic polymers may include poly(lactic-co-glycolic acid) (PLGA), Poly Lactic Acid (PLA), Polyethylene, Polyvinyl chloride (PVC), Polystyrene, Polypropylene (PP), Polyvinyl alcohol (PVA), Polycarbonate (PC), Nylon, Acrylonitrile butadiene styrene (ABS), etc.

In addition to the above-mentioned method, there is a method of filling a photocurable polymer into the recess or the engraving in the PDMS mold and then exposing the filled polymer to UV light to photocured the filled polymer to produce the drug-loaded portion.

Examples of various photocurable polymers may include SU-8 photoresist, Poly(ethylene glycol)dimethacrylate (PEGDMA), Bisphenol A glycidyl methacrylate (BISGMA), IP series resist, etc.

FIG. 5B shows another embodiment of a drug-loaded portion produced using hyaluronic acid (HA) as a material of the drug-loaded portion of the microneedle. HA is mixed with deionized water to make a solution which in turn is poured into the engraving in the PDMS mold, and a vacuum degassing process is performed thereon such that the recess in the PDMS mold is filled with HA. Afterwards, the HA is dried to fabricate the HA drug-loaded portion. Another method is to fill the PDMS mold with HA using a screen printer to make the drug-loaded portion. In addition to the HA used in one embodiment of the present disclosure, various naturally derived polysaccharides and protein polymers may be employed.

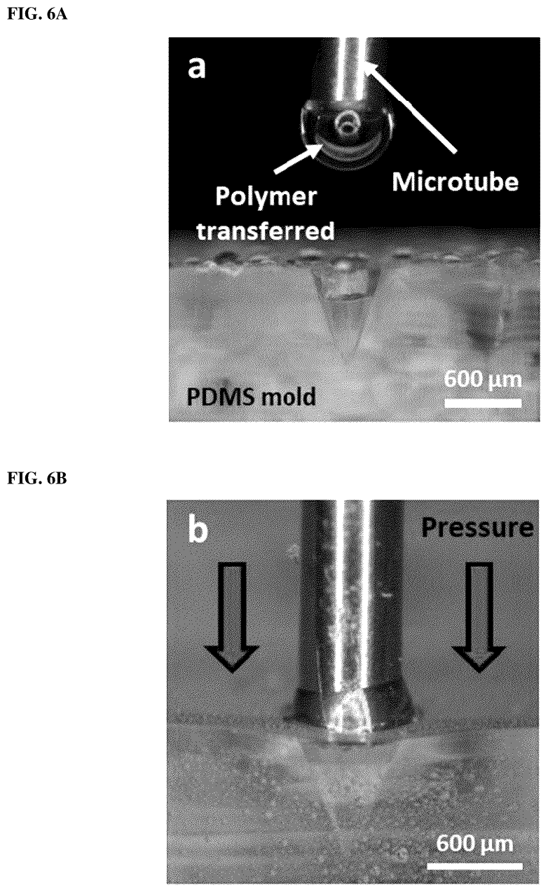

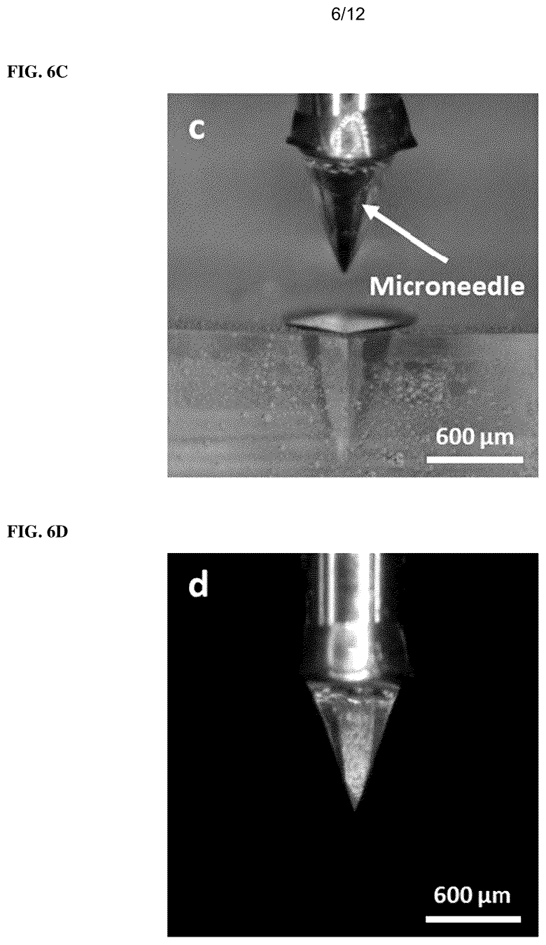

FIGS. 6A to 6D shows that a removable microneedle used in one embodiment of the present disclosure is manufactured using a pressure-assisted transfer molding technique using a microtube. The pressure-assisted transfer molding is a technique in which a polymer as a base material is transferred to the PDMS mold in which the drug-loaded portion has been formed using a rod as shown in FIG. 6A, and then contacts the drug-loaded portion as shown in FIG. 6B, and a pressure is applied to remove air bubbles trapped therein, and then a curing process is performed thereon, such that the drug-loaded portion and the base are coupled to each other as shown in FIG. 6C and FIG. 6D to produce the microneedle.

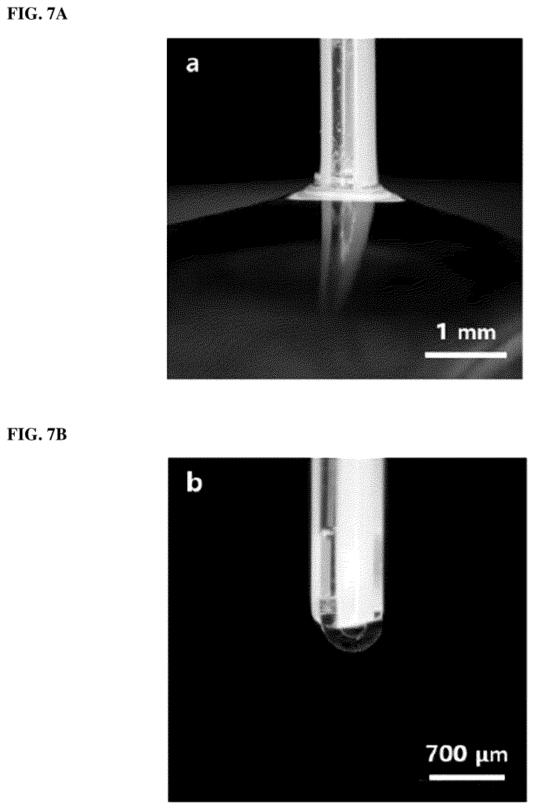

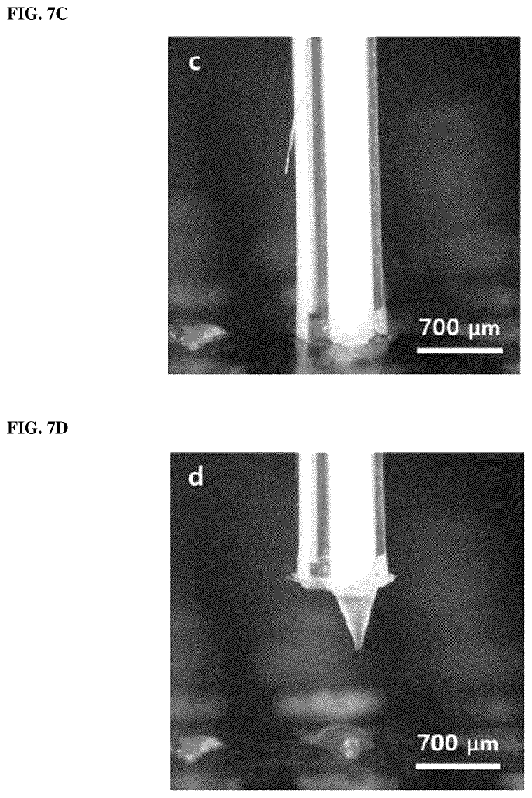

FIGS. 7A to 7D is a photograph of the microneedle structure manufactured according to an embodiment of the present disclosure. In one embodiment of the present disclosure, a microneedle with a removable anchor structure is manufactured using a microtube made of PTFE material in the pressure-assisted transfer molding manner. As mentioned above, the liquid polymer such as PLGA, HA, or SU-8 is transferred to the PDMS mold using the microtube made of the PTFE material as shown in FIG. 7A and FIG. 7B. Thereafter, the transferred polymer is brought into contact with the drug-loaded portion as shown in FIG. 7C, and then a curing process is performed thereon to produce a removable anchor structure microneedle as shown in FIG. 7D.

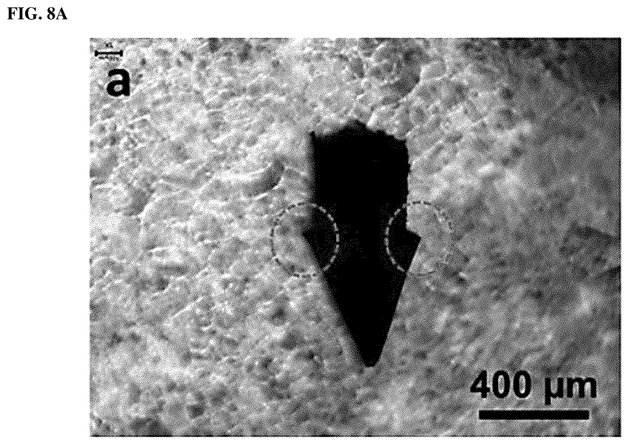

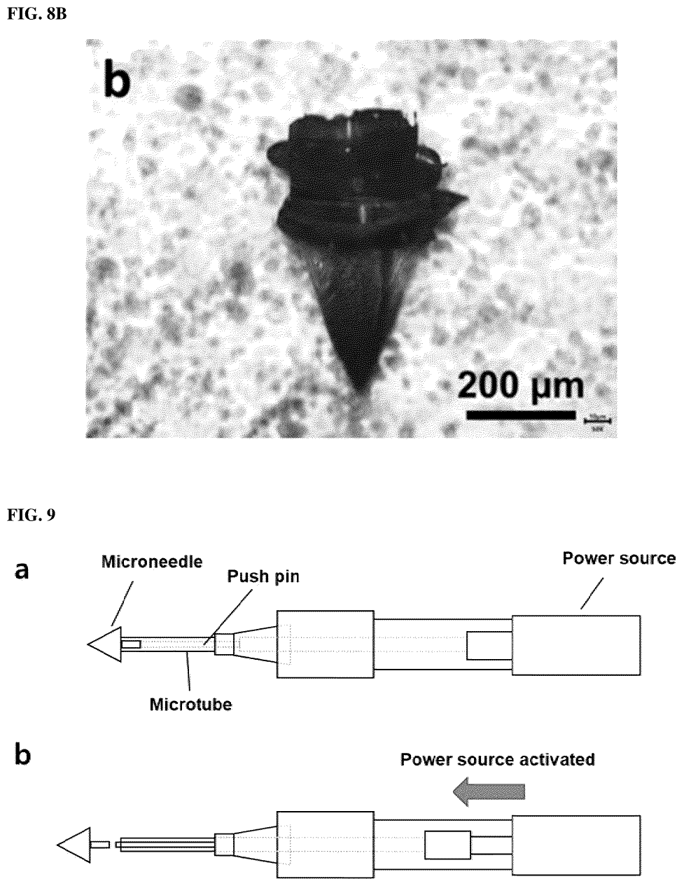

FIGS. 8A to 8B is a photograph showing the anchor structure of the microneedle structure according to an embodiment of the present disclosure. In accordance with the present disclosure, the shape of the microneedle has the anchor structure as shown in FIG. 8A and FIG. 8B to facilitate the removal of the drug-loaded portion after the microneedle has invaded into the skin. An outer diameter of the upper surface of the drug-loaded portion of the microneedle is larger than the inner diameter of the microtube, and a microneedle with a step structure is produced as shown in FIG. 8A. Due to this step structure, when the microneedle has been inserted into the skin, the step structure may be caught with the living tissue, allowing for more reliable removal thereof.

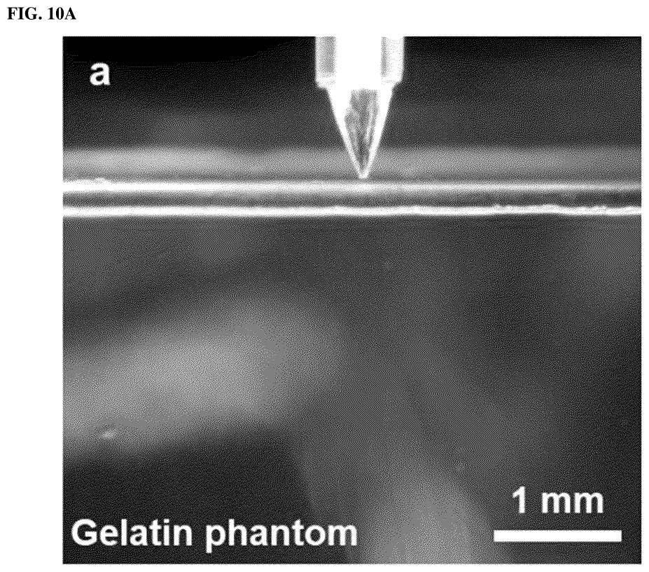

FIG. 9 is a diagram showing a removal applicator and a process in which the removable anchor structure microneedle of the present disclosure is pushed away from the applicator.

The microneedle removal system according to one embodiment of the present disclosure is manufactured using a push-pull solenoid as the power source. Referring to FIG. 9A, in one embodiment, a microneedle with a removable anchor structure manufactured in a pressure-assisted transfer molding method is attached to an end of the microtube, and a push pin and a push-pull solenoid are disposed on the back end of the microneedle. As shown in FIG. 9B, when the power source, that is, the push-pull solenoid in this embodiment works, a rod placed in front thereof is pushed, and the microneedle together with the rod is pushed and removed. It may be expected that various power sources may be used in addition to the push-pull solenoid used in one embodiment.

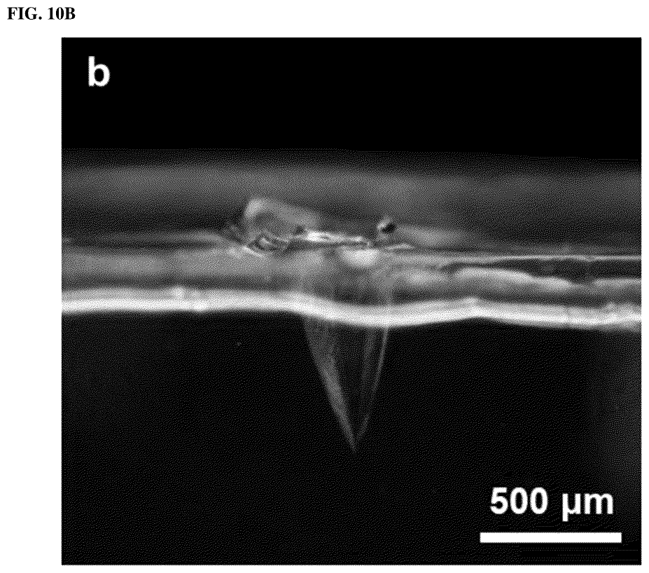

FIGS. 10A to 10B is a diagram showing that the removable microneedle with the removable anchor structure according to one embodiment of the present disclosure is removed using the removal applicator.

Referring to FIG. 10A and FIG. 10B, the removal and insertion of the microneedle with the removable anchor structure are identified using a gelatin tissue simulating model (Gelatin phantom) for skin simulation. As shown in FIG. 10A, the anchor structure removal applicator is placed at the desired insertion location in the gelatin tissue-simulating model, and the power source is activated to induce the microneedle to be removed and inserted. FIG. 10B is a diagram showing that the removable anchor microneedle has been inserted into the gelatin tissue-simulating model after the removal applicator is activated.

Although some embodiments of the present disclosure have been described above with reference to the accompanying drawings, the present disclosure may not be limited to some embodiments and may be implemented in various different forms. Those of ordinary skill in the technical field to which the present disclosure belongs will be able to appreciate that the present disclosure may be implemented in other specific forms without changing the technical idea or essential features of the present disclosure. Therefore, it should be understood that some embodiments as described above are not restrictive but illustrative in all respects.

What is claimed is:

1. A highly-removable microneedle structure comprising:
a tip tapered downwardly;
a base extending upwardly from an upper surface of the tip and having an area size in a plan view thereof smaller than an area size in a plan view of an upper surface of the tip; and
a tube surrounding the base and extending upwardly from the upper surface of the tip and beyond an upper surface of the base.

2. The highly-removable microneedle structure of claim 1, wherein the base is fitted into the tube.

3. The highly-removable microneedle structure of claim 1, wherein the tip or the base includes a delivery target drug.

4. The highly-removable microneedle structure of claim 1, wherein the tip or the base includes at least one selected from a group including a thermoplastic polymer; a photocurable polymer; a biocompatible polysaccharide polymer including hyaluronic acid, chitosan, and alginic acid; and a natural biomolecule including a protein or DNA.

5. The highly-removable microneedle structure of claim 1, wherein the tube includes at least one selected from a group including a polymer including PTFE, a metal, and a ceramic.

6. A microneedle device having a highly-removable microneedle structure, the microneedle device comprising:
a microneedle structure including:
a tip tapered downwardly;
a base extending upwardly from an upper surface of the tip and having an area size in a plan view thereof smaller than an area size in a plan view of an upper surface of the tip; and
a tube surrounding the base and extending upwardly from the upper surface of the tip and beyond an upper surface of the base; and
a microneedle pushing unit including:
a push pin extending upwardly from the upper surface of the base and beyond an upper surface of the tube; and
a pushing means capable of applying a pushing force to an upper end of the push pin,
wherein the microneedle device is configured such that when the pushing means applies the force to the upper end of the push pin, a lower end of the push pin applies the force to the base, so that a combination of the tip and the base is removed from the tube, the lower end of the push pin fixes the combination of the tip and the base into a skin, so that the tube is easily removed from the combination.

7. The microneedle device having the highly-removable microneedle structure of claim 6, wherein the base is fitted into the tube.

8. The microneedle device having the highly-removable microneedle structure of claim 6, wherein the tip or the base includes a delivery target drug.

9. The microneedle device having the highly-removable microneedle structure of claim 6, wherein the tip or the base includes at least one selected from a group including a thermoplastic polymer; a photocurable polymer; a biocompatible polysaccharide polymer including hyaluronic acid, chitosan, and alginic acid; and a natural biomolecule including a protein or DNA.

10. The microneedle device having the highly-removable microneedle structure of claim 6, wherein the tube includes at least one selected from a group including a polymer including PTFE, a metal, and a ceramic.

11. The microneedle device having the highly-removable microneedle structure of claim 6, wherein the pushing means applies the force to the push pin under at least one of a force from a human, an electromagnetic force, a spring force, a pneumatic pressure, and a hydraulic pressure.

12. A method for manufacturing a highly-removable microneedle structure, the method comprising:
providing a mold a recess defined therein, the recess having a tip shape tapered downwardly;
filling a curable delivery target substance solution into the recess;
providing a tube having an inner diameter smaller than an outer diameter of an upper surface of the tip shape;
contacting the one end of the tube with an upper surface of the delivery target substance solution received in the recess while filling the delivery target substance solution into one end of the tube and closing the other end of the tube; or applying a negative pressure to the other end of the tube.

13. The method for manufacturing the highly-removable microneedle structure of claim 12, wherein the delivery target substance solution includes a delivery target drug.

14. The method for manufacturing the highly-removable microneedle structure of claim 12, wherein the delivery target substance solution includes at least one selected from a group including a thermoplastic polymer; a photocurable polymer; a biocompatible polysaccharide polymer including hyaluronic acid, chitosan, and alginic acid; and a natural biomolecule including a protein or DNA,
wherein the delivery target substance solution is subjected to heat and thus is provided in a fluid state in which the delivery target substance solution can fill the recess.

15. The method for manufacturing the highly-removable microneedle structure of claim 12, wherein the tube includes at least one selected from a group including a polymer including PTFE, a metal, and a ceramic.
